(19) United States
(12) Patent Application Publication
(10) Pub. No.: US 2005/0222080 A1
(43) Pub. Date: Oct. 6, 2005

(54) Cycloglycans suitable to inhibit mammalian infection

(75) Inventors: Bernd Stahl, Rosbach (DE); Berndt Finke, Herford (DE); Joachim Schmitt, Hosbach (DE); Werner Goebel, Gerbrunn (DE); Jorg Slaghuis, Wurzburg (DE); Gunther Boehm, Echzell (DE)

Correspondence Address: BACON & THOMAS, PLLC, 625 SLATERS LANE, FOURTH FLOOR, ALEXANDRIA, VA, 22314, US

(73) Assignee: N.V. Nutricia, HM Zoetermeer (NL)

(21) Appl. No.: 10/502,059

(22) PCT Filed: Jan. 20, 2003
(86) PCT No.: PCT/EP03/00505

§ 371 Date: Aug. 2, 2004

(30) Foreign Application Priority Data
Feb. 1, 2002 (DE) 102 03 999.2

(51) Int. Cl. A61K 31/724; A61K 31/715

(52) U.S. Cl. 514/54; 514/58

(57) ABSTRACT

Described is the use of in particular homopolymer cycloglycans having a ring-shaped base structure of 2 to 40 monosaccharides in the ring, for preventing the invasion and infection of mammal cells by pathogens, and for combating diseases caused by such pathogens, and food compositions, dietetic compositions and pharmaceutical compositions containing these cycloglycans.

The invention relates to the use of in particular homopolymeric cycloglycans having a ring-shaped or annular, respectively base structure of 2 to 40 monosaccharides in the ring, for preventing the invasion and infection of mammalian cells by pathogens, and for combating diseases caused by such pathogens, and to food, dietetic and pharmaceutical compositions containing these cycloglycans.

The adhesion of pathogenic organisms as well as of cell-damaging substances to the surface of mammalian cells, is the first step and an essential prerequisite for an infection or a damage of the cell. The interaction between the pathogens and the cells is formed by a ligand-receptor relationship. In this ligand-receptor relationships or interactions, glycosidic structures play an important role.

One possibility of influencing such ligand-receptor relationships consists in blocking and/or structurally altering the respective receptors on the cell surface or the ligands.

Using specific test systems, At could be shown that various carbohydrate mixtures reduce or even completely prevent the adhesion of, for example, micro-organisms to the cell surface, cf.: Kunz, C; Rudloff, S. Acta Paediatr. 1993, 82, 903-912. Other substances such as the Lewis structures as the carbohydrate ligands of selectines (adhesion proteins in endothelial cells and lymphocytes) modulate the interaction of lymphocytes with the endothelium, for example, within the scope of rolling, homing and the invasion during inflammatory processes (Norman, K. E.; Anderson, G. P.; Kolb, H. C.; Ley, K,; Ernst, B. Blood 1998, 91, 475-483). A further important physiological role in conjunction with the fundamental cellular functions, as well as with specific functions such as cell adhesion, migration, chemotaxis, proliferation, apoptosis, neurite growth, is performed by the galactose-recognizing lectins, the galectins. (Cooper D N & Barondes S H, Glycobiology 1999 9 (10) 979-984). It could be shown for the nematode C. elegans that its galectin LEC-1 may bind various galactose-containing oligosaccharide derivatives of a different specificity. (Arata Y. Hirabayashi J. Kasai K, JBC, 2001:276, 5, 3068-3077). In a mouse model, the lethality of an experimental listeriosis could be reduced using galactose-specific lectins (Stoffel B., Beuth J., Pulverer G., Zentralbl. Bakteriol. 1996, 284:439-442). But the adherence of microorganisms to host cells may also be the trigger for signal cascades both in the exogenous pathogens and in the endogenous cells.

Another possibility consists in acquiring an influence on cellular processes on a molecular-biological level. This may lead to the fact that, for example, defence mechanisms are triggered in mammal cells, or in pathogenic microorganisms the expression of virulence mechanisms (e.g. disabling of virulence genes in bacteria by blocking of central regulators) is reduced or prevented. In this way, the expression of certain surface structures of pathogenic listeria, which are responsible for the invasion in host cells, may be impeded successfully by certain carbohydrates such as cellobiose (Park S F, Kroll R H, Mol Microbiol 1993, 8:653:661: WO-A 94/02586).

It is the object of the present invention to show a way how to reduce the invasion and infection of mammal cells by pathogens with the help of carbohydrates, and how to effectively combat diseases caused by such pathogens.

This object is solved by the teaching of the claims.

According to the invention, specific cycloglycans are used to solve the object of the invention. These cycloglycans are hereinafter called inventive cycloglycans.

According to the invention, both single inventive cycloglycans alone and several inventive cycloglycans in combination may be used. Furthermore, it is possible to use one inventive cycloglycan or several inventive cycloglycans or also a mixture of numerous inventive cycloglycans together with other carbohydrates not counting among the inventive cycloglycans in the form of a carbohydrate mixture.

The inventive cycloglyeans have 2 to 40 monosaccharides in the ring; these are monosaccharide units bound to each other, which here are simply called monosaccharides for the sake of better representation. These monosaccharides constitute a ring polymer. The inventive cycloglycans thus may have 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39 and 40 monosaccharides in the ring.

Preferably, the inventive cycloglycanes are made up of 6 to 40, particularly preferred of 6 to 20, and further particularly preferred of 6 to 8 monosaccharides.

Further preferred, the inventive cycloglycans are homopolymeric cycloglycans. In other words, the ring-shaped or annular, respectively, base structure has only monosaccharides of one kind or is made up of the same monosaccharides.

The ring of the cycloglycans preferably is made up of D-fructose, D-mannose, L-fucose, D-N-acetyl glucosamine, D-N-acetyl galactosamine, D-xylose, sialic acids (e.g. N-acetyl neuraminic acid), L-rhamnose, D-arabinose, D-allose, D-talose, L-idose, D-ribose, D-galacturonic acid, altrose, D-galactose and glucoses. These monosaccharides are bound to each other in a ring shape and constitute the ring of the in particular homopolymeric cycloglycans according to the invention.

In these cycloglycans, the glucosidic linkages both in the ring and in the cycloglycans linked to the ring and which will be described below in more detail, may be the following: α1-2, α1-3, α1-4, α1-6, α2-3, α2-6, α2-8, β1-2, β1-3, β1-4, β1-6, β2-1.

The bonds of the monosaccharides to each other preferably are α-glycosidic or β-glycosidic. Preferred inventive cycloglyeans are those having 6, 7 or 8 glucose units in the ring in an α-1-4-glycosidic bond. These include the α-cyclodextrins, β-cyclodextrins and γ-cyclodextrins and the derivatives thereof which are mentioned here.

Particularly preferred are the underivatized cyclodextrins. Hence it will be assumed, without being bound to this explanation, that it is the ring-shaped structure of the inventive cycloglycans that is responsible for the effects described within the framework of the present documents.

The inventive cycloglycans—as mentioned—may also be derivatized and namely by one or more monosaccharide group(s), disaccharide group(s) and/or other functional groups. The substitution may be present on the ring itself or on the free hydroxyl groups of the monosaccharides constituting this ring. In other words, one, two or more sugar units may among others be attached to the ring in a glycosidic bond.

Preferred derivatized cycloglycans are those, wherein one, two or all of the following criteria are met:
i) the cycloglycans are derivatized at one or more of the monosaccharides forming the ring by one or more of the following monosaccharide groups bound thereto in a glycosidic linkage: D-fructose, D-mannose, L-fucose, D-N-acetyl glucosamine, D-N-acetyl galactosamine, D-xylose, sialic acids, L-rhamnose, D-arabinose, D-allose, D-talose, L-idose, D-ribose, D-galacturonic acid, altrose, D-galactose and glucoses;
ii) the cycloglycans are derivatized at one or more of the monosaccharides forming the ring, by one or more of the following disaccharide groups bound thereto in a glycosidic linkage: lactose, maltose, sucrose and galacto-N-acetyl glucosamine;
iii) one or more of the OH groups of one or more of the monosaccharides forming the ring is or are substituted by an NH₂ group, SH group, phosphate group, sulfate group, nitrate group, alkyl group, hydroxyalkyl group or carboxyalkyl group. In the alkyl, carboxyalkyl and hydroxyalkyl groups, the alkyl residue preferably has 1 to 6 carbon atoms. The monosaccharides forming the ring thus may be derivatized, for example, by one or more methyl, ethyl, hydroxyethyl, carboxymethyl and hydroxypropyl groups, to name only a few;
iv) one or more of the OH groups as well as of the—if present—NH₂ and SH groups of the monosaccharides forming the ring are derivatized in the form of ethers, esters, amides and imines. These derivatives may for example be succinyl, acyl (in particular with 1 to 25 C-atoms, moreover in particular 1, 2, 3, 4, 5 or 6 C-atoms, e.g. acetyl) methylmalonic acid ester, phosphoglyceryl and phosphocholinyl derivatives. The acyl derivatives moreover include in particular those containing nutrtionally valuable saturated or unsaturated fatty acids with 12 to 22 C-atoms.

Further preferred inventive cycloglycans are the following:

Glycosyl-α-cyclodextrins, maltosyl-β-cyclodextrins, hydroxypropyl cyclodextrins, cyclofructines, cyclomannines, cyclogalactines and cycloaltrines.

The total number of the monosaccharides making up the molecule is 2-250, irrespective of whether these monosaccharides are within the ring or are bound to the ring and constitute derivatives.

This range of 2-250 is representative of all singe values within the range limits, and thus of the values 2, 3, 4, 5, 6, 7, 8, 9, 10, 11, 12, 13, 14, 15, 16, 17, 18, 19, 20, 21, 22, 23, 24, 25, 26, 27, 28, 29, 30 . . . 50, 51, 52, 53, 54, 55 . . . 70, 71, 72, 73, 74, 75, 76, 77, 78, 79, 80 . . . 100, 101, 102, 103, 104, 105, 106, 107, 108, 109, 110, 111, 112, 113, 114, 115, 116, 117, 118, 119, 120 . . . 137, 138, 139, 140, 141, 142 . . . 158, 159, 160, 161, 162, 163 . . . 179, 180, 181, 182, 183, 184 . . . 199, 200, 201, 202, 203, 204, 205, 206, 207, 208 . . . 219, 220, 221, 222, 223, 224, 225, 226 . . . 239, 240, 241, 242, 243, 244, 245, 246, 247, 248, 249, 250.

The inventive cycloglycans are known compounds or may be prepared according to known methods. Cycloglyeans of small rings (e.g. with only 2 monosaccharides in the ring) are described in: Armspach D., Gattuso G., Königer R., Stoddart J F, Cyclodextrins in: Bioorganic Chemistry: Carbohydrates. (S M Hecht ed.) Oxford Univ. Press New York 1999.

The inventive cycloglycans include the cyclodextrins wherein the ring is made up of the monosaccharide or the monosaccharide unit glucose. They exist in a natural way as an α-cyclodextrin, β-cyclodextrin and γ-cyclodextrin. These cyclodextrins preferably are used according to the invention. They are obtained enzymatically from starch by the activity of cyclodextrin-glucosyltransferases (CGTases ), a microbial enzyme (e.g. bacillus macerans). The differentiation into the three natural cyclodextrins is based on the number of the glucose molecules involved. α-cyclodextrin includes 6, β-cyclodextrin includes 7 and γ-cyclodextrin includes 8 glycopyranose units each bound into a ring in an α1-4-glycosidic linkage. Further cyclodextrins having larger molecules with usually up to 10 monosaccharide units, for example, are described in M J Playne & R. Crittenden, Commercial available oligosaccharides, Bulletin of the IDF 313, 1996, 10-22. Other cycloglycans, preferred β-1-2-linked cycloglycans, are to be found in the periplasmic space of various bacteria. Ring-shaped molecules consisting of up to 40 monosaccharide units which may be derivatized (in particular at the free hydroxyl groups), are, for example, described in: cf. Talaga P., Stahl B., Wieruszeski J.-M., Hillenkamp F., Tsuyumu S., Lippens G., Bohin J.-P, Cell-associated Glucans of Burkholderia solanacearum and Xanthomonas Campestris pv. Citri: a New Family of Periplasmic Glucans; Journal of Bacteriology 1996, 178, 8, 2263-2271.

The inventive cycloglycans may be prepared according to appropriate known methods in a chemical or enzymatic way, or in a combination of these two technologies. This combination takes place systematically from monosaccharide components or by modification of appropriate oligosaccharide raw materials. In the enzymatic syntheses, both transferases (Leloir or non-Leloir) and hydrolases (reverse hydrolysis or transglycosylation) are used. The enzymes in this case may be linked both freely or integratedly (e.g. membrane reactor) or covalently to a carrier (e.g. beads, chromatographic material or filtration membranes). It is also possible to use procaryontic or eucaryontic cells for the synthesis, insofar as these cells have suitable enzymes. With respect to further details of the composition of the inventive cycloglycans, reference is made, for example, to Carbohydrates in Chemistry and Biology (Editors Ernst, Hart, Sinay; Wiley VCH-Weinheim 2000, Vol. I-IV).

Further inventive cycloglycans are described in the following bibliographic references or may be prepared according to the methods described there: Armspach D., Gattuso G., Königer R., Stoddart J F, Cyclodextrins in: Bioorganic Chemistry: Carbohydrates. (S M Hecht ed.) p. 458, Oxford Univ. Press New York 1999.

Robyt J F, Cyclodextrins in: Essentials of Carbohydrate Chemistry (C R Cantor ed.) Springer, New York, 1998.

By way of additional information, reference is made to the following: if the inventive cycloglycans is only made up of 2 monosaccharides, then it is not substituted; on the contrary, it only consist of the ring of these two monosaccharides.

Surprisingly, it has now been found that the inventive cycloglycans at least reduce or even prevent the invasion and infection of mammalian cells by pathogens and may be used to combat diseases caused by such pathogens. These pathogens include invasive gram-positive and gram-negative pathogenic bacteria such as, for example, intracellular bacteria, in particular listeria, and pathogenic viruses, e.g. rotaviruses.

It has been found, for example, that the inventive cycloglycans may prevent the invasion and infection of mammalian cells by listeria, in particular Listeria monocytogenes. The results of the tests conducted clearly show that neither the process of phagocytosis as such, nor the replication of the ingested listeria is inhibited. Among other things, the cyclodextrins of the invention turned out to be particularly strongly inhibitory.

All in all, it could be ascertained that the inventive cycloglycans possess an anti-infectuous or inhibitory action with respect to an infection with listeria and salmonellas. The inventive cycloglycans can prevent the invasion of listeria on macrophage cell lines. Since the infection of a cell is often preceded by an adhesion and invasion of pathogens, a transferability exists to all of the pathogens, the infection of which proceeds in accordance with this listeria mechanism. Above all, these are salmonellas and E. coli.

Up to now, it was only known from the inventive cycloglycans that they are able to complex other substances and substance classes so as to improve the solubility behavior thereof. According to the invention, it was, however, noted that the cycloglycans posses anti-infectuous properties enabling these compounds to be used for prevention or therapy of an infection without necessitating the addition of further substances. If required, further active agents may of course also be useded together with the inventive cycloglycans.

The cycloglycans according to the invention may not only be used as free or unlinked cyclooligosaccharides, but may also be used bound to or immobilized on, e.g. adsorbed onto a carrier. This carrier may be a peptide/protein (e.g. BSA), a lipid (glycolipids, ceramide), a polymer or a biopolymer (e.g. carbohydrate dendrimer, polysaccharide, polyacrylamide) or any other aglykone.

The inventive cycloglycans, be it unbound or free cycloglycans or cycloglycans bound to a carrier, may be incorporated into various food, dietetic and pharmaceutical compositions. All of these compositions may be present in the form appropriate for the desired administration, and in particular in a fluid or solid form. The term “food compositions” used herein not only comprises the actual food composition but also food supplements, beverages and food compositions including infant and baby formulae. The term “baby or infant formulae” refers in particular to all artificially prepared formulae. “Artificial” means those food compositions which are produced from vegetable or animal but not from human origin. These food compositions may be administered to a human being or an animal in any desired way. This also includes administration as an infusion solution and a probe food into the stomach. The inventive cycloglycans, however, may also be added to natural milks, in particular animal milks.

The inventive cycloglycans may, for example, be added as admixtures or additives to the following products, although this enumeration is not conclusive: milk and milk products, infant and babyfood formulations, chocolate bars, yoghurt drinks, cheese, sausage and meat products, anabolic food, probe food and products for pregnant women and for immuno-suppression.

Apart from the inventive cycloglycans, further carbohydrates may also be present in the inventive composition, so that the inventive compositions comprise a carbohydrate mixture, with the inventive cycloglycans representing a part of this carbohydrate mixture.

The inventive cycloglycans may also be administered in the form of a pharmaceutical composition alone or together with one or several additional active agent(s). These compositions may, for example, be formulated as a tablet/capsule. For the formulation of such pharmaceuticals, usual adjuvants, carriers, auxiliary agents, diluents, moisturizing agents, thickening agents, flavoring agents, sweetening agents, etc. may be used.

The pharmaceutical compositions may be administered in any usual way to a patient (i.e. human and animal). However, for the sake of convenience, they will be compositions suited for oral, lingual, nasal, intestinal, bronchial, vaginal, topical (skin and mucosa) and per os administration and formulated to suit the kind of administration.

The foods, dietetic compositions and pharmaceutical compositions containing at least one inventive cycloglycan, may be used among other things for preventing and treating infections of the gastrointestinal tract, e.g. in case of listerioses, of the blood system, the respiratory passages, the urogenital tract, as well as of the nasopharynx, and for protecting endothelia, epithelia and mucosa. Thus, they may be applied topically to the skin or may also be used on mucous membranes. These mucous membranes include nasal, intestinal, bronchial and vaginal mucous membranes. Thus, the inventive cycloglycans may, for example, be added to a mouthwash. All age groups, ranging from new born babies up to elderly people, may be mentioned as target groups for the inventive cloglycans. Special fields of application are the protection and the treatment of pregnant women, sick persons, debilitated and elderly people, for whom the pevention e.g. of a listeriosis is of particular importance.

Exemplary dietetics and pharmaceuticals containing at least one inventive cycloglycan are listed below. These are the following inventive cycloglycans: α-cyclodexthn, cyclodextrin, β-cyclodextrin, γ-cyclodextrin cyclofructines (DP 6-8), clomannines (DP 6-8), cyclogalactines (DP 6-8), cycloaltrines (DP 6-8), periplasmic cycloglycans (DP 6-25). For the sake of simplicity, these cycloglycans will be termed “cycloglycan” in the examples. This term is representative for each of the above-mentioned inventive cycloglycans and the mixtures thereof.

### EXAMPLE 1

For preparing sachets, in each case 100 mg of cycloglucan are mixed in a dry state with 990 mg of maltodextrin, and then are packed in sachets. These sachets are administered three times per day during meals,

### EXAMPLE 2

A known medicinal food (i.e. Milupa® HN 25, balanced diet) in the form of a bead product containing 18.8 g of protein, 8.6 g of fat, 62.8 g of carbohydrates, 3.3 g of minerals and vitamins, is admixed in a composition known per se with cycloglycan in such an amount that 50 mg of cycloglycan are contained in 100 g of the finished bead product.

For the composition of a liquid medicinal food, 100 ml of the known medicinal food Milupa HN 25 liquid (2.3 g of protein, 1.6 g of fat, 8.5 g of carbohydrates, 37 g of minerals and vitamins) are admixed with 7 mg of cycloglycan.

### EXAMPLE 3

A Product for Pregnant Women

An effervescent tablet (final weight 4.15 g) (Neovin® from Milupa) is prepared in a manner known per se by admixing 200 to 500 mg of cycloglucan. One tablet per day is dissolved in 150 ml water and swallowed.

### EXAMPLE 4

A Product for the Elderly and Debilitated Persons

A balanced pulverized medicinal food (Dilsana® from Milupa) containing 22.5 g of protein, 7.7 g of fat, 60.8 g of carbohydrates, 5.4 g of minerals and vitamins is prepared in a manner known per se by incorporating 100 mg to 1000 mg cycloglucan per 100 g of powder. Up to 3×50 g per day of the food are dissolved in 150 ml water and administered.

### EXAMPLE 5

Tea

100 g of an instant tea powder prepared in the usual manner are mixed with 2 g of cycloglycan. 3.8 g of tea powder are dissolved in 100 ml of hot water, and administered three times per day,

### EXAMPLE 6

A protein-adapted infant milk formulation (Aptamil® from Milupa) containing 11.8 g of protein, 56.9 g of carbohydrates, 24.9 g of fat, 2.5 g of minerals and vitamins and 45 mg of taurine are prepared in the usual manner in the form of a bead product, which is mixed with 100 mg to 1000 mg of cycloglycan per 100 g of infant milk formulation.

1-18. (canceled)

19. A method for reducing or preventing the invasion and infection of mammalian cells by pathogens, and for combating diseases caused by such pathogens comprising administering to a mammal an effective amount of cycloglycans having a ring-shaped base structure of 2 to 40 monosaccharides in the ring, which is unsubstituted or may be derivatized at the monosaccharides forming said ring, by one or more monosaccharides(s) or disaccharide(s) and/or one or more functional group(s), wherein the total number of the monosaccharides making up the molecule of the cycloglycans is 2 to 250, and said cycloglycans may also be bound to an inert carrier or may be immobilized thereon.

20. The method of claim 19, characterized in that the cycloglycans are homopolymeric cycloglycans and/or the ring of the cycloglycans is made up of 6 to 40, and in particular 6 to 20 monosaccharides.

21. The method according to claim 19, characterized in that the ring of the cycloglycans is made up of D-fructose, D-mannose, L-fucose, D-N-acetyl glucosamine, D-N-acetyl galactosamine, D-xylose, sialic acids, L-rhamnose, D-arabinose, D-allose, D-talose, L-idose, D-ribose, D-galacturonic acid, altrose, D-galactose and glucoses,

22. The method according to claim 19, characterized in that one, two or all of the following criteria are met:
i) the cycloglycans are derivatized at one or more of the monosaccharides forming the ring by one or more of the following monosaccharide groups bound thereto in a glycosidic linkage: D-fructose, D-mannose, L-fucose, D-N-acetyl glucosamine, D-N-acetyl galactosamine, D-xylose, sialic acids, L-rhamnose, D-arabinose, D-allose, D-talose, L-idose, D-ribose, D-galacturonic acid, altrose, D-galactose and glucoses;
ii) the cycloglycans are derivatized at one or more of the monosaccharides forming the ring, by one or more of the following disaccharide groups bound thereto in a glycosidic linkage: lactose, maltose, sucrose and galacto-N-acetyl glucosamine;
iii) one or more of the OH groups of one or more of the monosaccharides forming the ring is or are substituted by an NH₂ group, SH group, phosphate group, sulfate group, nitrate group, alkyl group, hydroxyalkyl group or carboxyalkyl group;
iv) one or more of the OH groups as well as of the—if present—NH₂ and SH groups of the monosaccharides forming the ring are derivatized in the form of ethers, esters, amides and imines.

23. The method according to claim 19, characterized in that the linkage of the monosaccharides in the ring is α-glycosidic or β-iglycosidic, with the β-glycosidically linked monosaccharides in particular being periplasmic glycans.

24. The method according to claim 19, characterized in that the cycloglycans have 6, 7 or 8 monosaccharides and in particular glucose units in the ring.

25. The method according to claim 19, characterized in that the cycloglycans are the following:
α-cyclodextrin, β-cyclodextrin, γ-cyclodextrin; cyclofructines, cyclo-mannines, cyclogalactines and cycloaltrines, which may be derivatized in the manner described in claim 22.

26. The method according to claim 25, characterized in that the derivatized cycloglycans are glucosyl-α-cyclodextrins, maltosyl-β-cyclodextrins and hydroxypropyl cyclodextrins.

27. The method according to claim 19, characterized in that the carrier is a peptide, a protein, a lipid, a lipoid, a polymer or a biopolymer.

28. The method according to claim 19, characterized in that the cycloglycans are incorporated into a fluid or solid food composition, a dietetic composition or a pharmaceutical composition for administration to a human or an animal, or serve for preparing such a composition.

29. The method according to claim 29, characterized in that the composition serves for an oral, lingual, nasal, bronchial, vaginal, topical (skin and mucosa) and per os administration, for an administration by means of a probe into the stomach of a human or an animal, or for an administration as an infusion.

30. The method according to claim 19, characterized in that the cycloglycans are administered once daily in an amount of at least 1 mg per kg of body weight to a human or an animal.

31. The method according to claim 19, for the prevention and treatment of infections of the gastrointestinal tract, blood system, respiratory passages, urogenital tract, as well as the nasopharynx, in particular in case of listerioses.

32. The method according to claim 19, characterized in that the pathogens are invasive gram-positive and gram-negative pathogenic bacteria, in particular listeria, and pathogenic viruses.

33. A food composition, dietetic composition or pharmaceutical composition containing a cycloglycan described in claim 19, or several such cycloglycans.

34. A composition according to claim 33, characterized in that it may contain a further carbohydrate or more further carbohydrates, which are not a cycloglycan as described in claim 19, a further active agent or several further active agents and/or a further ingredient, which is known and suited for the corresponding composition, or more of such ingredients, wherein in the case of a pharmaceutical composition a usual auxiliary agent or several usual auxiliary agents, including diluents, moisturizing agents, thickening agents, flavoring agents, sweetening agents and carriers, may be present,

35. A method of reducing or preventing the invasion and infection of mammal cells by pathogens, and of combating diseases in humans and animals caused by such pathogens, characterized in that at least one cycloglycan according to claim 19 is administered to a human or an animal, in particular in such an amount that at least 1 mg of cycloglycan per kg of body weight is supplied to the human or the animal once daily.
